(19) United States
(12) Patent Application Publication
(10) Pub. No.: US 2020/0170545 A1
(43) Pub. Date: Jun. 4, 2020

(54) Mobile Real-Time Breath Ketone And Exhaled Gas Detector

(71) Applicant: NGageIT Digital Health, Inc., Toronto (CA)

(72) Inventor: Raj Reddy, Burlington (CA)

(21) Appl. No.: 16/524,947

(22) Filed: Jul. 29, 2019

Related U.S. Application Data
(60) Provisional application No. 62/711,603, filed on Jul. 29, 2018.

(51) Int. Cl. A61B 5/083; A61B 5/087; G01N 33/00; A61B 5/08

(52) U.S. Cl. CPC A61B 5/0836; A61B 5/082; G01N 33/004; A61B 5/087

(57) ABSTRACT

Systems and methods for mobile real-time breath ketone and exhaled gas detectors are described. For example, a device comprises an opening, providing a flow path through which a person breathes, a sensor, providing a ketone signal corresponding to a concentration of a respiratory component in exhalations of the person and including metabolic data, wherein the concentration of the respiratory component is associated with a level of ketone bodies in blood of the person, a display, and an electronic circuit, receiving the ketone signal and the metabolic data and providing a visual indication of the ketone signal and the metabolic data on the display.

### CROSS-REFERENCE TO RELATED APPLICATION(S)

This application claims priority to and the benefit of U.S. Provisional Application Patent Ser. No. 62/711,60, filed Jul. 29, 2018, the entire disclosure of which is hereby incorporated by reference.

### TECHNICAL FIELD

This invention relates to the respiration analysis of a mammal, in particular to the detection of ketones and other gases in the exhaled breath of a person, and further relates to exercise monitoring and diet management as well as an extension to breath detection of diseases by classifying exhaled volatile gas patterns.

### BACKGROUND

The metabolism of fat, in particular the breakdown of triglycerides, leads to the accumulation of ketone bodies in the blood. These ketone bodies comprise acetone, acetoacetic acid and beta-hydroxybutyric acid. In the blood, acetone exists in the form of acetoacetate. The concentration of acetone in exhaled breath is well correlated with the concentration of acetone in the blood. Therefore, breath analysis provides valuable information about the physiological status and metabolic processes present in a person.

### SUMMARY OF THE INVENTION

Disclosed herein are implementations of mobile real-time breath ketone and exhaled gas detectors.

In a first aspect, a device comprises an opening, providing a flow path through which a person breathes, a sensor, providing a ketone signal corresponding to a concentration of a respiratory component in exhalations of the person and including metabolic data, wherein the concentration of the respiratory component is associated with a level of ketone bodies in blood of the person, a display, and an electronic circuit, receiving the ketone signal and the metabolic data and providing a visual indication of the ketone signal and the metabolic data on the display.

In a second aspect, a respiratory analyzer system, comprises a flow path operable to receive and pass respiratory gases, the flow path having a first end in communication with a respiratory connector and a second end in communication with a source for the respiratory gases, the respiratory connector configured to be supported in contact with a subject so as to pass exhaled gases as the subject breathes, the flow path comprising a flow tube mouthpiece through which the exhaled gases pass, and a sensor chamber attached to smartphone disposed between the flow tube and the first end, the sensor chamber being a concentric chamber surrounding one end of the flow tube, and a ketone sensor, providing a ketone signal correlated with a ketone concentration in the exhaled gases passing through the flow path.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates an exemplary method by depicting a flow diagram for detecting ketones by training a Neural Network to classify an exhaled gas.

FIG. 2 illustrates exemplary flow diagram showing the flow path for exhaled gas through a nanoparticle ketone sensor attached to a mobile smartphone device and the wireless transmission of correlated data from the mobile device to the cloud and the display of the data.

FIG. 3 illustrates an exemplary method for detecting ketones from breath using a nanoparticle sensor and shows a plausible ketone sensor fabrication using NanoFibers.

FIG. 3A illustrates a schematic diagram of Plausible sensor fabrication which can be NanoFibers or CarbonNanoFibers (CNF) with embedded selective NanoParticle which can be Nano Metal Oxide (MOX) selectively trapping an exhaled gas which can be Acetone.

FIG. 4 is an illustration is an illustration of a sample breath ketone detection system in accordance with a preferred embodiment of the invention.

### DETAILED DESCRIPTION

Human breath is mainly composed of nitrogen, oxygen, carbon dioxide, water vapor and inert gases. In addition, thousands of volatile organic compounds (VOCs) are exhaled at very low concentrations that can be selectively used to classify and identify certain diseases from the breath. Novel sensors such as those utilizing nanoparticles which can be mesoporous and selective enzyme systems immobilized on nano-composite structural materials with specific selection and sensitivity capabilities can be combined with smartphone technology and cloud computing to create a vastly improved method for respiratory analysis.

In the present invention, we describe a system, device, and respiratory analyzer to sense the respiration for a person or any mammal that comprises a mouthpiece or flow capture path through which the person breathes. The device can include a nanoparticle composite exhaled gas sensor which can be a ketone sensor, providing a ketone signal related to the concentration of respiratory components correlated with a level of ketone bodies in exhalations of the person; a display or correlated display on a smartphone device; and an electronic circuit, receiving the ketone signal and the metabolic data which can be streamed to the internet cloud by a smartphone for processing by a computerized system such as a neural network, and providing a visual indication of the metabolic rate and the ketone signal to the person on the display.

The respiratory analyzer may also include a metabolic rate meter, providing a metabolic rate for the person. The respiratory analyzer can be used in an improved exercise management program for the person. Further, the respiratory analyzer can be customized to detect other Volatile Gas (VG) patterns exhaled by persons or mammals in their breath that are specifically correlated with disease such as Lung cancer, Breast Cancer and Digestive Cancers or correlated with levels of exhaled anesthetic gases used for medical procedures such as surgery. Diagnostic breath testing would be extremely attractive, because it is totally noninvasive and painless to the individual, having no undesirable side effects. Real-time breath testing by simply exhaling into an instrument would be especially useful, because the data could be immediately available to the clinician, allowing swift treatment decisions and reducing the number of visits to the clinic.

Weight control is an important goal of a large proportion of the U.S. population. Conventional weight control programs typically allow a restricted range of caloric intake per day, with some allowance made for activity levels. However, even though caloric intake is monitored with some precision, the effects of physical activity are not measured in a quantitative way. Physical activity is an important component of weight control programs for several reasons. It can be used to reduce the body fat proportion of a person. It can help reduce the fall in resting metabolic rate of a person on a restricted caloric intake. Activity is initially fueled by blood sugar, but after a sustained period of activity a person will start to metabolize fat. Few people on weight control programs are aware of how much exercise is required to start the fat metabolizing process, and they may not be fully aware of the beneficial effects of activity on their resting metabolic rate.

The present invention describes an apparatus (i.e., respiratory analyzer) for the measurement of breath ketones. The ketone is usually assumed to be acetone. Aldehydes such as acetaldehyde may also be detected by the methods described below.

The present invention describes a nanoparticle based sensor apparatus based on nanocomposites, nanotubules or nanofibers with immobilized substances upon them such as biological enzymes such as laccase or custom nanoparticles which are tuned to select for specific gases and substances that re exhaled in the breath. In the case of Aldhehydes or ketones they are selectively detected by laccase and or the custom nanoparticles which have a fixed porosity designed to adhere to selected exhaled gases such as ketones or aldehydes such as acetone thereby sensing the selected exhaled gas such as acetone. For electrochemical sensing materials like carbon nanofibers or CarbonNanoTubes or polymeric nanofibers are synthesized according to the selected gases to be detected. Nanoparticles are defined as a solid colloidal particles having size in the range from 10 to 1000 nm, which offers many benefits to larger particles such as increased surface-to-volume ratio and increased magnetic properties.

Over the last few years, nanoparticles are gaining fast momentum in different biomedical applications such as targeted drug delivery (fast and sustain), hyperthermia, photoablation therapy, bioimaging and biosensors (in the form of bar-coding). Duet to excellent chemical stability, non-toxicity, biocompatibility, high saturation magnetization and high magnetic susceptibility, iron oxide nanoparticles have dominated applications in drug delivery, hyperthermia, bioimaging, and cell labeling and gene delivery. Besides this, mesoporuous silica nanoparticles, superpapramagnetic nanoparticles, core-shell structure (inorganic-inorganic, inorganic-organic, organic-inorganic and organic-organic such as PLA-PEG), silver nanoparticles and gold nanoparticles can also used for above mentioned applications. Even nanotubes and hydrogel may also act as good biomedical sensing agents.

Resting metabolic rate can also be estimated from the Harris-Benedict equation, as discussed by Karkanen in U.S. Pat. No. 5,839,901, and using metabolic rate meters comprising gas sampling techniques and differential pressure based flow rate sensors, for example as disclosed by Acorn in U.S. Pat. No. 5,705,735.

In some cases, it can be useful to monitor the composition of inhaled gases, for example when administering gases to the patient such as anesthetics, nitric oxide, medications, and other treatments, monitoring pollutants or environmental effects, for a person respiring with the assistance of a ventilator, or for persons using breathing apparatus. For convenience, the analysis of exhaled gases will be discussed, though the embodiments described can also be used for analysis of inhaled gases.

Ketone detection can also be achieved using a hand-held nanoparticle based respiratory analyzer which could be attached to a smartphone or be used as a stand alone mobile device. A person holds the analyzer to their mouth, and breathes through a mouthpiece. Exhaled air is conveyed along a flow tube. The exhaled air may be dried by conventional means, e.g. using silica gel. Preferably, the drying process should not remove substantial proportions of the gas component of interest from the expired air. Volatile organic compounds such as acetone can be adsorbed or selectively trapped at the molecular level on a nanoparticle surface, and detected and quantified by the selective electrochemical nanoparticle sensor system. Selectively permeable membranes may also be used to allow nitrogen, oxygen, and possibly carbon dioxide to exit a detector device, while concentrating volatile organics such as ketones for detection by any appropriate method.

In devices previous to this invention, exhaled air vented from a respiratory analyzer can be further analyzed, for example by routing the exhaled air to an analytical device such as a mass spectrometer, chromatography device, calorimeter, or other instrument. For example, ketones and other volatile organic compounds in exhaled gases can be detected by gas chromatography. The exhaled air is passed through a flame and combustion reactions are detected using characteristic optical emission and/or absorption lines. Oxygen, carbon dioxide, nitrogen, and rare gases are not combusted by a flame, but in the breath such as ketones are combusted. In U.S. Pat. No. 4,114,422, Hutson describes a hydrogen flame ionization scheme to detect acetone in the breath, which can be advantageously combined with an indirect calorimeter.

In U.S. Pat. No. 4,758,521, Kundu describes adsorption of ketones onto solid pellets, and chemical detection using a nitroprusside salt in one solid matrix, with an amine coupled to a second solid matrix. Chemical detection methods such as this may be incorporated into the disposable part of the GEM (gas exchange monitor). Colorimetry may be used to detect the onset of significant levels of fat burning by the person's metabolic processes; such threshold-ty[0019] Data may be transferred from the ketone sensor to other devices such as a smartphone by direct attachment, portable computer, interactive television component (e.g. set-top box, web-TV box, cable box, satellite box, etc.), desk-top computer, wireless phone, etc. via Bluetooth protocol radio communication, IR communication, transferable memory sticks, wires, or other electromagnetic/electrical methods. Data may also be transferred to a remote computer via a communications network such as the Internet. In a preferred embodiment, data is transferred to a Smartphone by direct connection. Data can be transferred to the internet cloud via wireless communication such as cellular or Bluetooth pe detection does not need an updateable real-time ketone.

The following example illustrates how breath ketone measurements can be used in an improved weight loss program involving an exercise component. A person is equipped with an activity sensor (e.g. pedometer, accelerometer) and starts an activity routine (e.g. running on the spot). A nanoparticle sensor with additional ketone sensing capability is used to monitor the person's oxygen intake rate and hence metabolic rate; and also to detect the attainment of a certain acetone level in the person's breath, indicating the onset of fat catabolism. The data is transferred to a smartphone and to the internet cloud securely. Data transfer to the smartphone may be using IR communication, Bluetooth protocol wireless communication, direct connection or through the transfer of a memory stick (such as those manufactured by Sony or SanDisk). The data can be used to create a model of the person's physiological response to exercise.

During a daily exercise routine, a signal from the activity sensor is transferred to the smartphone then to the cloud, preferably using the Bluetooth. The smartphone is then used to provide quantitative feedback to the person on the benefits of the exercise. For example, the smartphone may be used to indicate the calories burned, the time the exercise must continue for the onset of fat burning, or an estimate of fat grams burned. This level of feedback is a great improvement over previous weight control/exercise programs, and a very powerful motivational factor for the person to continue with the exercise.

The following example illustrates a diet and exercise control program for a person suffering from diabetes. The person carries a smartphone and has a glucose sensor transmitting blood glucose levels to the smartphone using a wireless transmission protocol such as Bluetooth. Dietary intake is entered into the smartphone. The smartphone is used to track dietary intake and blood sugar levels, estimate possible future deviations of blood sugar from an acceptable range, and provide warnings and advice to the person. Indirect calorimetry is used to determine the metabolic rate of the person. An activity sensor is used to provide a signal correlated with physical activity. These data are transmitted to the smart phone, preferably using Bluetooth. Breath ketone sensing is used to detect the onset of the dangerous condition of ketoacidosis.

A system for warning a person of the onset of ketoacidosis comprises a smartphone application carried by the person, a blood glucose sensor, and a respiratory analyzer (which device functions of indirect calorimeter and respired volatile organics detector, in two way communication using wireless communication. Data may also be transferred to the internet cloud for further analysis from the smartphone using wireless communication.

The respiratory analyzer may be attached onto a smartphone directly or combined with mobile technology into a portable unitary device. Also, the ketone sensing device may be combined or be separate from the calorimeter.

The following example relates to exercise management. A person exercising carries a portable ketone analyzer that includes a tube that is breathed through and a nanoparticle ketone detector disposed on one wall of the tube. The device may be small, such as the size of a human finger. The exerciser may periodically blow through the device to determine whether they are burning fat. Alternatively, the device may prompt the user to periodically blow, or may signal that analysis is required after a certain period of time has passed. Also, a separate exercise monitor may wirelessly signal the analyzer that a breath should be analyzed after a certain set of conditions are met. The analyzer may wirelessly communicate the results back the an exercise monitor, may give a confirmation of results such as by a chime indicating fat burning, or may store the results versus time onto a non-volatile memory device. The data can be streamed from the smartphone in real-time to the internet cloud for further analysis.

Hence, a method for encouraging exercise in a person comprises: monitoring a metabolic rate of a person during an exercise, and hence correlating the exercise with metabolic rate; detecting the presence of organic compounds in the breath of the person, indicative of fat metabolizing processes in the person, and hence determining the effect of exercise on fat burning; providing feedback to the person during future repetition of the exercise, in terms of the effect of the exercise on metabolic rate and fat burning whereby the person is encouraged to continue exercising by the provision of the feedback.

Embodiments of the present invention can be used to detect numerous volatile organic compounds in the breath, which include ketones such as acetone, aldehydes such as acetaldehyde, hydrocarbons including alkanes such as pentane, alkenes, and fatty acids, and other compounds. Embodiments of the present invention can further be used to detect nitric oxide, ammonia, carbon monoxide, carbon dioxide, and other components of exhaled breath. Respiration components produced by certain bacteria within the mouth, stomach, and intestinal tract can also be detected using embodiments of the present invention.

A respiratory analyzer according to the present invention can be combined with gas flow sensors so as have the capabilities of a spirometer. The improved spirometer is useful for detecting respiratory components such as nitric oxide diagnostic of asthma and other respiratory tract inflammations. The combination of respiratory component analysis and flow rate analysis is helpful in diagnosing respiration disorders.

Certain persons desire a diet low in carbohydrates and high in protein. A respiratory analyzer according to the present invention can be used to detect respiration components indicative of success in following such a diet.

Hence, an improved respiratory analyzer for a person, comprises: a flow path, through which the person breathes; a metabolic rate meter, providing metabolic data correlated with the metabolic rate of the person; a ketone sensor, providing a ketone signal correlated with a concentration of respiratory components in exhalations of the person, wherein the respiratory components are correlated with a level of ketone bodies in the blood of the person; a display; and an electronic circuit, receiving the ketone signal and the metabolic data, and providing a visual indication of the metabolic rate and the ketone signal on the display. The metabolic rate meter can comprise a pair of ultrasonic transducers, for example using the density of exhaled air to determine oxygen and carbon dioxide concentrations in exhaled air.

The metabolic rate meter can comprise a pair of ultrasonic transducers or nanoparticle flow sensors or microelectronic flow sensors, for example using the density of exhaled air to determine oxygen and carbon dioxide concentrations in exhaled air. The metabolic rate meter may comprise a flow rate sensor, and an oxygen sensor and/or a carbon dioxide sensor.

Embodiments of the ketone sensor are discussed in detail below. The ketone sensor can, for example, comprise a nanoparticle sensor mechanism or array to select for a particular exhaled compound such as ketones or acetone.

Novel nanoparticle based gas sensor's greatly enhance the selectivity and sensitivity of sensors that detect selected exhaled gases such as ketones or acetones. During designing and formulating such smart and versatile nanoparticles it is mandatory to have a look on commercially viable technique. There are various methods for the synthesis of nanoparticles using top-down (Mechanical Attrition, Nanolithography, Etching, Physical vapor deposition (PVD), Chemical vapor deposition (CVD)) and bottom-up approach (Sol-gel, Solvothermal, Sonochemical method, Microwave-assisted synthesis, Reduction in Solution, Template synthesis, Co-precipitation, Biosynthesis). Considering the control over the size, bottom-up approach is a choice of researchers and designers of nanoparticles. The sensors described herein were based on organically or inorganically functionalized nanomaterials that fulfill the stringent requirements of breath testing: the nanosize allows the implementation of very sensitive and reliable gas sensors, the adjustability of the chemical and physical properties allows optimal sensing of disease-specific VOC or VG patterns in humid atmospheres, and the ease of fabrication renders production reasonably cost effective.

During periods of restricted calorie input, the concentration of ketone bodies and fatty acids increase, whereas the concentration of glucose will fall. Hence, the detection of ketone bodies in the blood or urine, or their manifestation in the exhaled breath of a person, is indicative of successful adherence to a weight control program.

In a weight control program, a person will often attempt to achieve a body weight loss through restricted calorie intake. However, weight loss can arise through a variety of mechanisms, such as fat loss, muscle loss, and water loss. Conventional weight control programs often neglect the actual mechanism by which weight is lost. Further, breath testing could provide a totally new, comprehensive detection and screening method for digestive cancers, which can affect in the entire digestive system: esophagus, stomach, small intestine, colon, rectum, anus, liver, pancreas, gallbladder and biliary system. Digestive cancers belong to the most widespread and deadly human cancers. Colorectal cancer and stomach cancer, for example, are the second leading causes of cancer deaths in the USA and worldwide, respectively. Early presymptomatic detection is paramount in the management of digestive cancers, improving prognosis and treatment outcome. Endogenous cancer-specific VOCs are released from the cancer cells and/or metabolic processes that are associated with cancer growth whereby different cancers emit different types and/or amounts of molecules. These VOCs are transported with the blood to the alveoli of the lung from where they are exhaled as measurable odorants. Therefore, cancer does not only have a smell, but, different cancers have different smells.

In U.S. Pat. No. 4,970,172, Kundu describes a method of measuring acetone concentration in the breath, by extracting a sample volume of breath, and allowing acetone in the breath to interact with matrix materials containing a nitroprusside salt and an amine. However, sampling methods fail to provide a real-time determination of breath ketone level.

In U.S. patent application Ser. No. 09/630,398 and international application WO01/8554, Mault et al. describe an indirect calorimeter comprising an oxygen sensor and an ultrasonic flow meter. However, this device cannot detect aldehydes and ketones in exhaled breath.

Other ketone detection methods are known in the art, for example as described by Kundu in U.S. Pat. Nos. 5,174,959, 5,071,769, 4,970,172, 4,931,404; and U.S. Pat. No. 5,834,626 to DeCastro et al. However, there is no disclosure in these patents of a device for real-time monitoring of ketone levels by breath analysis. Further, in some cases impractical detection methods that utilize a Gas Exchange Monitor (GEM) used to measure the oxygen consumption of a person and hence their metabolic rate. The GEM comprises a bi-directional ultrasonic flow-meter and an oxygen sensor which uses the fluorescence quenching of a film by oxygen molecules with the addition radiation emission and or emitters to measure exhaled gases in particular ketones. This system is not designed to directly attach to a mobile smartphone and presents safety and use issues due to the use of radiation emission technology. New smart sensor technology is needed. [0009] During exercises of escalating intensity, the metabolism of fat causes ketone levels in the breath to increase.

During a restricted calorie diet, the rate of fat loss is correlated with breath acetone concentration, as disclosed by Kundu in U.S. Pat. No. 4,970,172. Hence, mobile monitoring of acetone levels in the breath via a smartphone can be used to provide valuable information on exercise programs and weight loss programs.

Embodiments described herein detect and classify certain exhaled gases from a person or mammal in a fluid medium or breath sample of a user and/or patient by a nanoparticle sensor which transmits data from a smartphone mobile wireless device to the cloud for processing which can be by a neural network based processor or computerized system. The substances or exhaled gases of interest are detected by the system using electronic and/or electromechanical sensors. The sensors convert the detection of certain substances such as ketones in the exhaled breath into electrical signals which are conveyed to a pattern recognition system, such as neural network, and a result is generated. Accordingly, a method and system in accordance with the present invention provides breath analysis with an accessible mobile device (e.g., smartphone) to determine valuable information about the physiological status and metabolic processes present in a person.

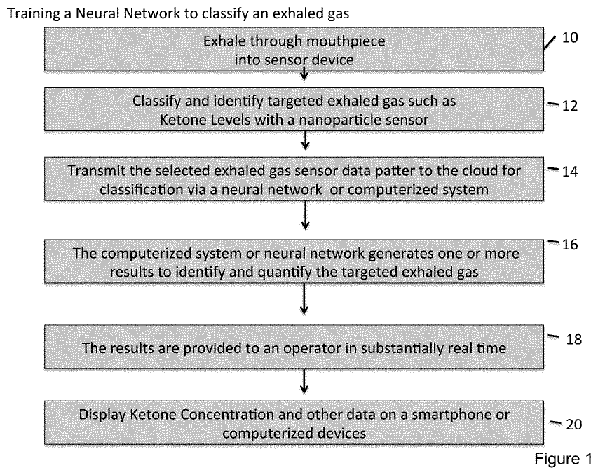

FIG. 1 illustrates an exemplary method for classifying an exhaled gas using a preferred embodiment. The method starts with training of a neural network, for example, using known gases through a nanoparticle based sensor (per step 10). Once the neural network is trained, it is deployed (per step 12). The deployed system receives one or more selected exhaled gases using a sensor or sensor group (per step 14). The received exhaled gases are processed using the neural network or computerized system which, in a preferred embodiment, is an artificial neural network (per step 16) and one or more results are generated. The results provide identification of exhaled gases based on received exhaled gases, or vapors or by identifying the unique electronic sensor derived signal pattern of the exhaled gases that are correlated with the underlying substance (per step 18). These results are provided to an operator in substantially real-time (per step 20).

As used herein real-time refers to an event or a sequence of steps, such as are executed by a processor that are perceivable by a user or observer at substantially the same time that the event is occurring or that the steps are being performed. By way of example, if the neural network of FIG. 1 receives an exhaled gas, the system produces a result at substantially the same time that the exhaled gas was sensed. This real-time processing can input to the neural network and further associated with the processing of data by the have some time delay associated with converting sensed exhaled gas to electrical signals for neural network; however, any such delay is less than 1 minute and typically no more than a few seconds.

A preferred embodiment of an electronic exhaled gas sensing apparatus is useful for detecting exhaled gases substances which can be ketones such as acetone.

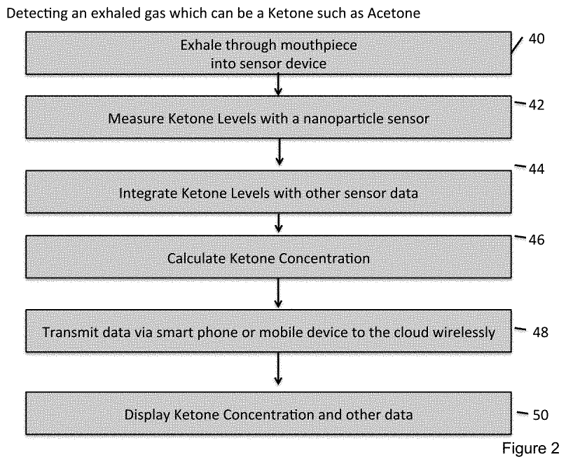

For example, this embodiment can be used for real-time site assessment and monitoring activities associated with diet and weight loss as well as monitoring and detection of ketones in diabetes. FIG. 2 illustrates an of a field measurement system capable of detecting and classifying exhaled gases such as ketones such as Acetone associated within a breath sample of a user and/or patient who is on a diet or weight control program or who is diabetic. A mouth piece 40 which is connected to a sensing instrument module [electronic breath sensor device] 42 and linked wirelessly 44 to a neural network 46 collects a breath sample of patient or user 48 which detects and displays the unique fingerprint or exhaled gas profile of that substance or gas 50 the sensing device which can be attached to a smartphone wireless platform to send data to the cloud wirelessly for assessment.

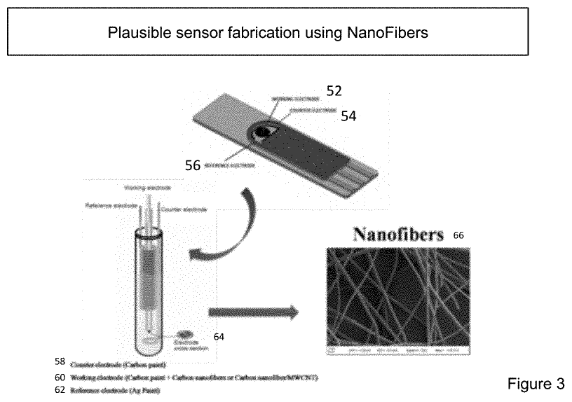

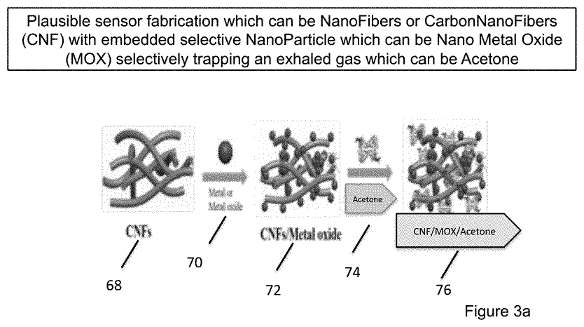

FIG. 3A preferred embodiment of an electronic exhaled gas sensing apparatus includes a plausible sensor using nanomaterials which can be nanofibers is useful for detecting exhaled gases substances which can be ketones such as acetone. The sensor includes a working electrode 52 and a counter electrode 54 and a reference electrode 56. In more detail, the exhaled gas sensor includes counter electrode 58 which can be made from a conducting paint which can be carbon paint and a working electrode 60 which can be made from a conducting paint which can be connected to a bed of carbon nanofibers or carbon nanofibers with carbon nanotubules which can be multi-walled 62 and a reference electrode 56 which can be made from a conducting paint such as a silver (Ag) material. The electrode cross section 64 can be fabricated from a bed of nanofibers 66 which can be embedded with sensing enhancing nanoparticles FIG. 3a for purposes such as selecting specific gas electrical fingerprint electrical signal patterns. FIG. 3a shows a preferred embodiment of a plausible sensor fabrication which can be NanoFibers or CarbonNanoFibers (CNF) 68 with embedded selective NanoParticle which can be Nano Metal Oxide (MOX) 70 selectively trapping an exhaled gas in a CNF/MOX matrix 72 which exhaled gas can be Acetone 74 which produces a complex whereby the CNF/MOX matrix is embedded with the trapped exhaled gas which can be Acetone 76 leads to a unique electrical fingerprint signal for the trapped gas which can be used for identification purposes.

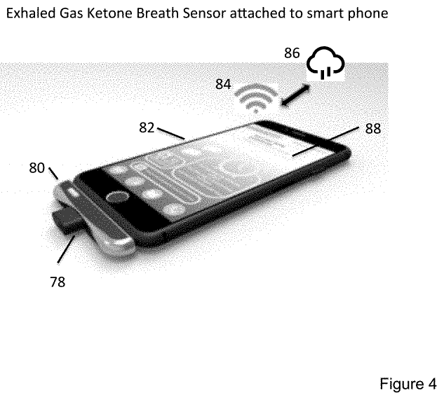

FIG. 4 is a preferred embodiment of an electronic exhaled gas sensing apparatus attached to a smartphone which includes a plausible sensor which can be NanoFibers or CarbonNanoFibers (CNF) with embedded selective NanoParticle which can be Nano Metal Oxide (MOX) selectively trapping an exhaled gas which can be Acetone which is used for identifying, quantifying and classifying selected exhaled gases. The device can allow users to exhale gas through a mouthpiece 78 which causes the gas to flow through and over an exhaled gas sensor such as described in FIG. 3 and FIG. 3a which can then identify and quantify the gas an unique electrical signal fingerprint which is sent through the smart phones computerized 82 wireless system 84 to the internet cloud 86 which is then processed through the clouds computerized identification system which can be a neural network and the processed identified exhaled gas signal is then returned to the 88 mobile smartphone device or computerized system display as a visual display of the identified exhaled gas.

As shown by the illustrated embodiments herein, the artificial neural network based breath sensor system is capable of being trained to detect substantially any identifiable exhaled or inhaled gas. Embodiments of the invention are therefore applicable to essentially any industry or application where automated detection and classification of exhaled gases or inhaled anesthetic gas types or correlated gases is desired.

In an implementation, a nanoparticle respiratory analyzer for a person having a metabolic rate and blood is provided. The nanoparticle respiratory analyzer comprises a flow path, through which the person breathes; a sensor, providing an exhaled volatile gas signal correlated with a concentration of a respiratory component in exhalations of the person, wherein the concentration of the respiratory component is correlated with a level of which are Volatile Organic Compounds (VOC) comprised of volatile gases (VG) in the blood of the person; a display; an electronic circuit, receiving the VG and the metabolic data, and providing a visual indication the VG signal on the computerized display; VG's signal patterns which are standardized in collection; and analysis are specifically correlated with certain correlated with corresponding disease states or exhaled anesthetic gases that can be identified using the nanoparticle VG sensor.

In an implementation a method for improving exercise management for a person performing an activity is provided. The method includes providing a wearable activity monitor connected to a smartphone or mobile computerized device; providing a metabolic rate meter connected to the smartphone or mobile computerized device; providing a respiratory analyzer having a nanoparticle ketone sensor attached to the smartphone or mobile computerized device; monitoring an activity signal from the activity monitor connected to the smartphone or mobile computerized device or integrated wearable electronic monitor; monitoring a metabolic rate signal from the metabolic rate meter attached to the smartphone phone or mobile computerized device, wherein the metabolic rate signal is correlated with a metabolic rate of the person; monitoring a ketone signal from the nanoparticle ketone sensor, wherein the ketone signal is correlated with a concentration of ketone bodies in blood of the person; and correlating the activity signal with the metabolic rate signal and the ketone signal, wherein the activity signal is used to determine a metabolic rate; and an estimate of fat burning for the person during an exercise, allowing improved exercise management.

In this implementation, a device that is attached to the smartphone or mobile computerized device comprises the metabolic rate meter and the ketone sensor. The method can identify unique VG's specially correlated with the following diseases that have a unique identified VG gas pattern: Gastric Cancer exhibits at least five distinguishable volatile organic compounds which the method in Claim 14 sensor system can identify (2-propenenitrile, 2-butoxy-ethanol, furfural, 6-methyl-5-hepten-2-one and isoprene) which are significantly elevated in patients with GC and/or peptic ulcer, as compared with less severe gastric conditions. The method can identify unique VG's specially correlated with the following diseases that have a unique identified VG gas pattern: Lung Cancer and typical VOCs that have been observed in the breath samples of lung cancer patients which the method in Claim 14 sensor system can identify, inter alia, (decane, benzene, aldehydes and branched aldehydes). The method can identify the unique VOC's comprised of VG's specially correlated with the following diseases that have a unique identified VG gas pattern and VOC patterns which identify different types of cancers from breath samples, including lung, breast, colorectal, prostate, head-and-neck, stomach and liver-cancer, as well as kidney disease and neurodegenerative diseases. The method can identify the unique VOC's comprised of VG's specially correlated with the following inhaled gases such as anesthetics which could be propafol.

In some cases, it can be useful to monitor the composition of inhaled gases, for example when administering gases to the patient such as anesthetics, nitric oxide, medications, and other treatments, monitoring pollutants or environmental effects, for a person respiring with the assistance of a ventilator, or for persons using breathing apparatus. Therefore, the method and systems provided by the present invention can also be used for analysis of inhaled gases such as propafol from the breath for non-invasive monitoring of intravenous drugs and control of anesthesia.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures.

The claims should not be read as limited to the described order or element unless stated to that effect. Therefore, all embodiments that come within the scope and spirit of the following claims and equivalents thereto are claimed as the invention.

What is claimed:

1. A device, comprising:
an opening, providing a flow path through which a person breathes;
a sensor, providing a ketone signal corresponding to a concentration of a respiratory component in exhalations of the person and including metabolic data, wherein the concentration of the respiratory component is associated with a level of ketone bodies in blood of the person;
a display; and
an electronic circuit, receiving the ketone signal and the metabolic data and providing a visual indication of the ketone signal and the metabolic data on the display.

2. The device of claim 1, wherein the sensor comprises a metabolic rate meter.

3. The device of claim 2, wherein the metabolic rate meter is a nanoparticle flow rate sensor.

4. The device of claim 3, wherein the nanoparticle flow rate sensor includes a nanoparticle oxygen sensor.

5. The device of claim 3, wherein the nanoparticle flow rate sensor includes a nanoparticle carbon dioxide sensor.

6. The device of claim 1, wherein the sensor is any of a nanoparticle sensor and a sensor array which captures exhaled breath through a part of the flow path.

7. A respiratory analyzer system, comprising:
a flow path operable to receive and pass respiratory gases;
the flow path having a first end in communication with a respiratory connector and a second end in communication with a source for the respiratory gases, the respiratory connector configured to be supported in contact with a subject so as to pass exhaled gases as the subject breathes, the flow path comprising a flow tube mouthpiece through which the exhaled gases pass, and a sensor chamber attached to smartphone disposed between the flow tube and the first end, the sensor chamber being a concentric chamber surrounding one end of the flow tube; and
a ketone sensor, providing a ketone signal correlated with a ketone concentration in the exhaled gases passing through the flow path.

8. The respiratory analyzer of claim 7, wherein the ketone sensor comprises a nanoparticle sensor.

9. The respiratory analyzer of claim 7, wherein the ketone sensor comprises a nanoparticle sensor or sensor array that is correlated with the ketone concentration.

10. The respiratory analyzer of claim 7, further comprising:
a flow rate sensor.

11. The respiratory analyzer of claim 10, wherein the flow rate sensor comprises a pair of nanoparticle or microelectronic or nanoelectronic flow rate sensors.
